(19) United States
(12) Patent Application Publication
(10) Pub. No.: US 2025/0352037 A1
(43) Pub. Date: Nov. 20, 2025

(54) AIR AND WATER VALVE FOR AN ENDOSCOPE

(71) Applicant: Boston Scientific Scimed, Inc., Maple Grove, MN (US)

(72) Inventors: Jenny Dandin, Worcester, MA (US); Henry Stock, Boston, MA (US); Colby Harris, Norfolk, MA (US)

(73) Assignee: Boston Scientific Scimed, Inc., Maple Grove, MN (US)

(21) Appl. No.: 19/205,109

(22) Filed: May 12, 2025

Related U.S. Application Data
(60) Provisional application No. 63/647,254, filed on May 14, 2024.
(60) Provisional application No. 63/760,245, filed on Feb. 19, 2025.

(51) Int. Cl. A61B 1/00

(52) U.S. Cl. CPC A61B 1/00068

(57) ABSTRACT

Devices, systems, and methods for an air and water valve assembly for a medical device. The valve assembly includes a valve cap and valve stem with a central lumen that fits within the internal valve well of a valve body. Two wiper seals and a one-way seal surround the valve stem. The valve stem's external diameter changes along its length in correspondence with the changing internal diameter of the valve well, such that that each of the seals extend a similar distance to obstruct gas or water as required.

### CROSS-REFERENCE TO RELATED APPLICATIONS

This application claims the benefit of U.S. Provisional Patent Application Ser. No. 63/760,245 filed on Feb. 19, 2025 and U.S. Provisional Patent Application Ser. No. 63/647,254 filed on May 14, 2024, the disclosures of which are incorporated herein by reference.

### TECHNICAL FIELD

This disclosure relates generally to valve assemblies and methods, and particularly to air and water supply valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a valve assembly includes a valve body having an internal valve well; a valve cap having a gas hole; a valve stem connected to the valve cap and configured to translate within the valve well between an upper position and a lower position; and upper and lower wiper seals extending circumferentially around the valve stem.

The valve body has a gas inlet passage, a gas outlet passage, a water inlet passage, and a water outlet passage each connected to the internal valve well. The internal valve well has five chambers. The first chamber opens into the gas outlet passage. The second chamber is below the first chamber, has a smaller diameter than the first chamber, and opens into the gas inlet passage. The third chamber is below the second chamber, has a smaller diameter than the second chamber, and opens into the water outlet passage. The fourth chamber is below the third chamber and has a smaller diameter than the third chamber. The fifth chamber is below the fourth chamber, has a larger diameter than the fourth chamber, and opens into the water inlet passage.

The valve stem has two gas inlets; and a central lumen extending from the gas inlets in the valve stem to the gas hole in the valve cap.

The upper wiper seal has first and second wipers positioned such that, when the valve seal is in the upper position, the first wiper contacts the internal wall of the first chamber of the valve well to obstruct gas flow through the first chamber, and when the valve seal is in the lower position, the second wiper contacts the internal wall of the second chamber of the valve well to obstruct gas flow through the second chamber.

The lower wiper seal has first and second wipers positioned such that, when the valve seal is in the upper position, the second wiper contacts the internal wall of the fourth chamber of the valve well to obstruct water flow through the fourth chamber, and when the valve seal is in the lower position, the first wiper contacts the internal wall of the fourth chamber of the valve well to obstruct water flow through the fourth chamber.

Alternatively or additionally to any of the examples above, the valve assembly can further include a one-way seal extending circumferentially around the valve stem, the one-way seal contacting the internal wall of the valve well to obstruct gas flow downwards into the second chamber while allowing gas to flow upwards.

Alternatively or additionally to any of the examples above, the one-way seal can be positioned in contact with a contour of the valve well between the first and second chambers of the valve well when the valve stem is in the upper position, the one- way seal obstructing gas flow from the first chamber into the second chamber.

Alternatively or additionally to any of the examples above, the one-way seal can be a flexible seal formed of a material with a durometer less than the durometer of a material forming the valve stem.

Alternatively or additionally to any of the examples above, the one-way seal may be configured to contact and seal against a vertical portion of the internal wall of the valve well at the second chamber.

Alternatively or additionally to any of the examples above, the upper wiper seal can be a flexible seal formed of a material with a durometer less than the durometer of a material forming the valve stem.

Alternatively or additionally to any of the examples above, the lower wiper seal can be a flexible seal formed of a material with a durometer less than the durometer of a material forming the valve stem.

Alternatively or additionally to any of the examples above, the valve stem can be injection-molded thermoplastic.

Alternatively or additionally to any of the examples above, the upper and lower wipers of the upper wiper seal and the upper and lower wipers of the lower wiper seal can all be of equal width.

Alternatively or additionally to any of the examples above, the valve stem can include portions having varying diameters, wherein the diameters of the portions vary according to the varying diameters of the first, second, third, and fourth chambers of the valve well.

Alternatively or additionally to any of the examples above, the valve assembly can include a valve collar configured to attach to the valve body, the valve collar remaining stationary relative to the valve body when the valve stem moves between the upper and lower positions; and a spring member connected to the valve collar and the valve cap, the spring member biased to move the valve cap upward to return the valve stem to the upper position from the lower position.

Alternatively or additionally to any of the examples above, the valve collar can include a plurality of latches sized and positioned to press against the valve body when the valve collar is attached to the valve body.

Alternatively or additionally to any of the examples above, the valve collar can be injected-molded thermoplastic

Alternatively or additionally to any of the examples above, the spring member can be formed of a material with a durometer less than the durometer of a material forming the valve collar

As another example, an endoscopic medical device includes an endoscopic probe; and the valve assembly of any of the above examples, the valve assembly coupled to the endoscope for use in an endoscopic procedure.

Alternatively or additionally to any of the examples above, the endoscopic medical device can further include an endoscopic operating handle including the valve body of the valve assembly.

Alternatively or additionally to any of the examples above, the one-way seal may be positioned in contact with a vertical inner wall of the valve well between the gas inlet passage of the valve body and the gas outlet passage of the valve body.

In another example, a valve for a medical device may comprise a valve stem configured to translate within a valve well between a first configuration and a second configuration, the valve stem comprising a proximal opening, a gas inlet, and a central lumen extending from the gas inlet to the gas escape hole, a one-way seal extending circumferentially around the valve stem at an axial location along the valve stem distal of the proximal opening and proximal of the gas inlet, wherein the one-way seal is configured to contact a vertical inner wall of a valve well between a gas inlet of the valve well and a gas outlet of the valve well.

Alternatively or additionally to any of the examples above, the valve may further include an upper wiper seal extending circumferentially around the valve stem at an axial location proximal of the one-way seal, the upper wiper seal comprising first and second wipers and a lower wiper seal extending circumferentially around the valve stem at an axial location distal of the one-way seal, the lower wiper seal comprising first and second wipers.

Alternatively or additionally to any of the examples above, the valve may further include a valve cap having a gas hole aligned with the proximal opening.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts perspective and exploded views of an illustrative supply valve;

FIG. 3B depicts a perspective view of an illustrative valve stem and illustrative valve seals for the valve stem;

FIGS. 3C and 3D depict top and bottom perspective views of an illustrative valve collar;

FIGS. 3E and 3F depict top and bottom perspective views of a one-way seal;

FIG. 3G depicts a cross-section view of the one-way seal depicted in FIGS. 3E and 3F;

FIG. 4A depicts a plan view of an illustrative supply valve and valve body;

FIG. 4B depicts a schematic cross-sectional view of the valve body depicted in FIG. 4A;

FIG. 4C depicts a schematic cross-sectional view of the supply valve and valve body depicted in FIG. 4A;

FIG. 5A depicts a schematic cross-sectional view of an illustrative supply valve and an illustrative valve body, with the supply valve in a first configuration;

FIG. 5B depicts a schematic cross-sectional view of the illustrative supply valve and the illustrative valve body depicted in FIG. 5A, with the supply valve in a second configuration; and

FIG. 5C depicts a schematic cross-sectional view of the illustrative supply valve and the illustrative valve body depicted in FIG. 5A, with the supply valve in a third configuration;

FIG. 6A depicts a schematic cross-sectional view of an illustrative supply valve and an illustrative valve body, with the supply valve in the first configuration or the second configuration;

FIG. 6B depicts a schematic cross-sectional view of the illustrative supply valve and the illustrative valve body depicted in FIG. 6A, with the supply valve in the third configuration;

FIGS. 7A and 7B depict schematic top and bottom perspective views, respectively, of an illustrative one-way valve; and

FIG. 8 depicts a schematic cross-sectional view of the illustrative one-way valve depicted in FIGS. 7A and 7B.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

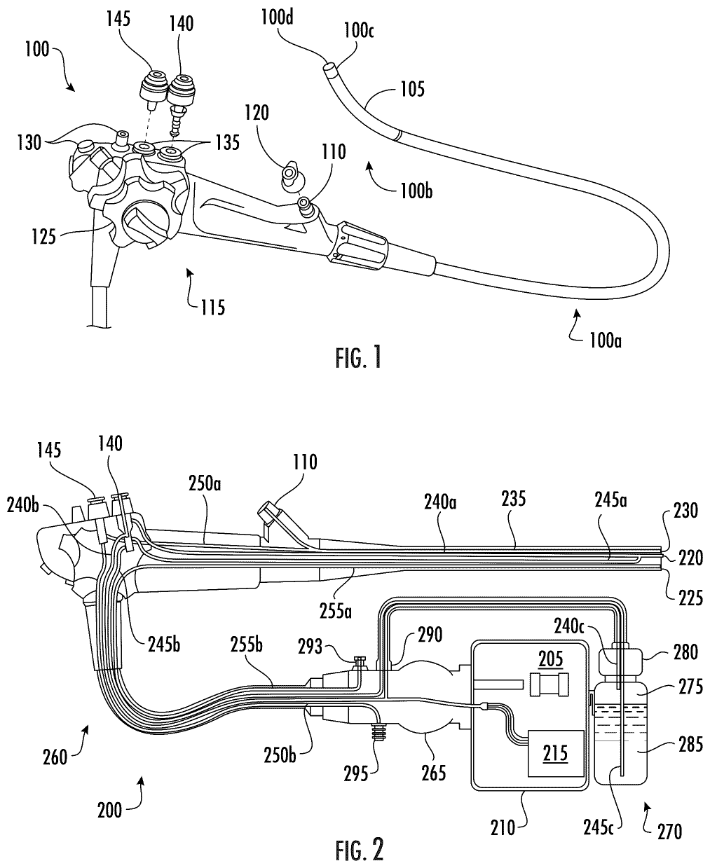

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as a gas feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

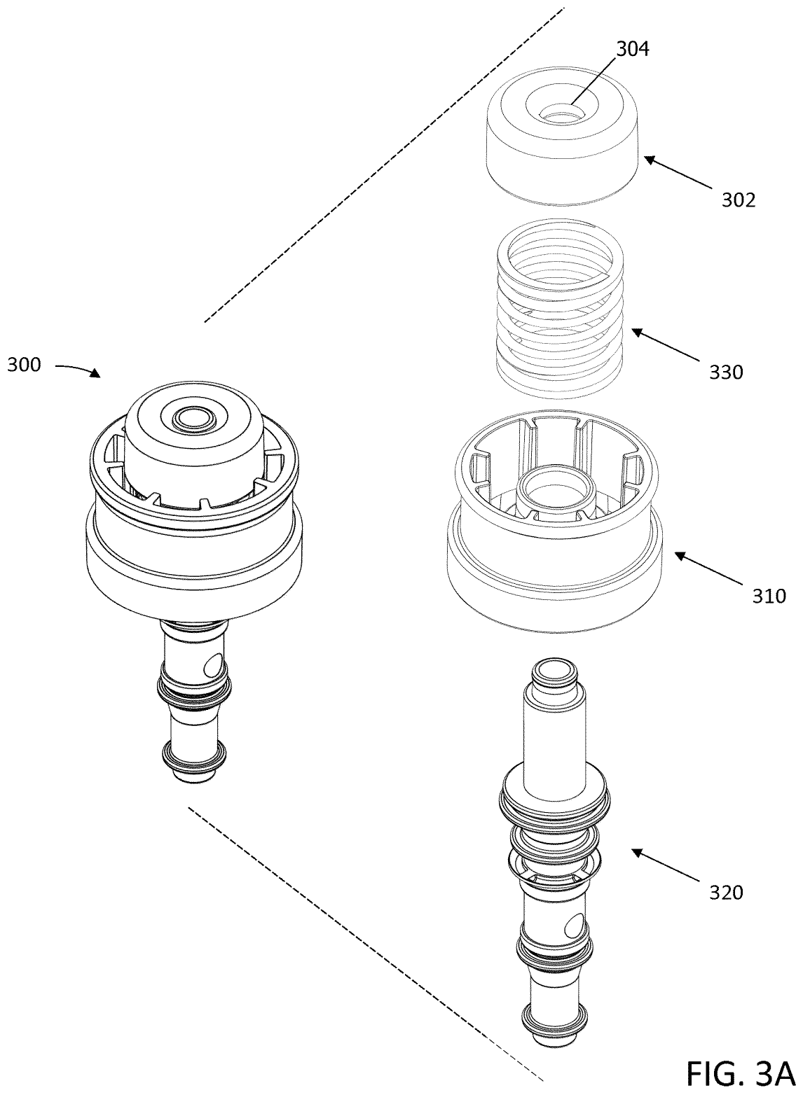

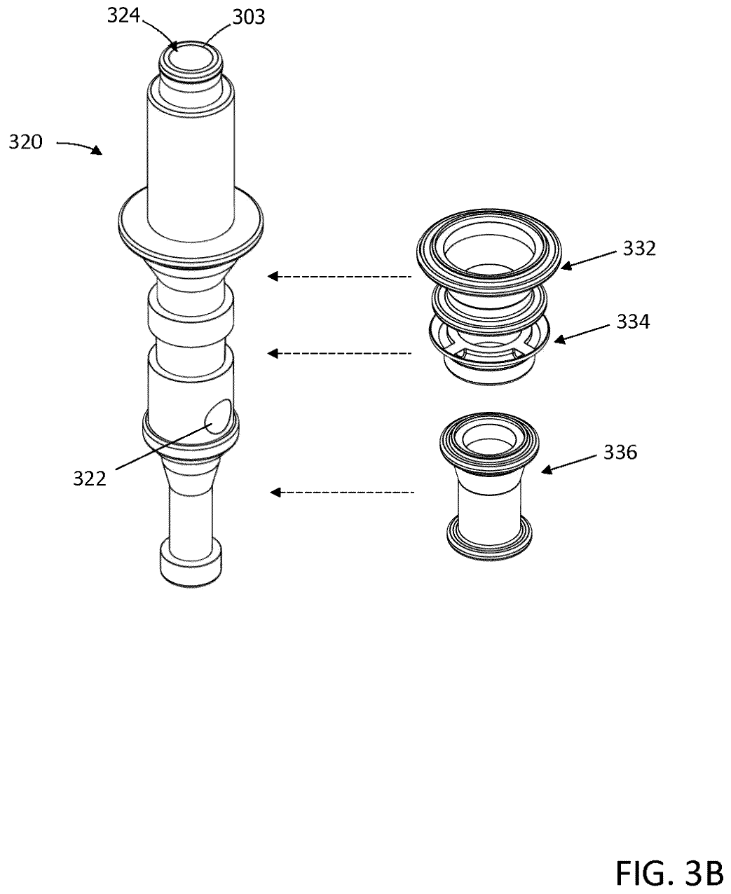

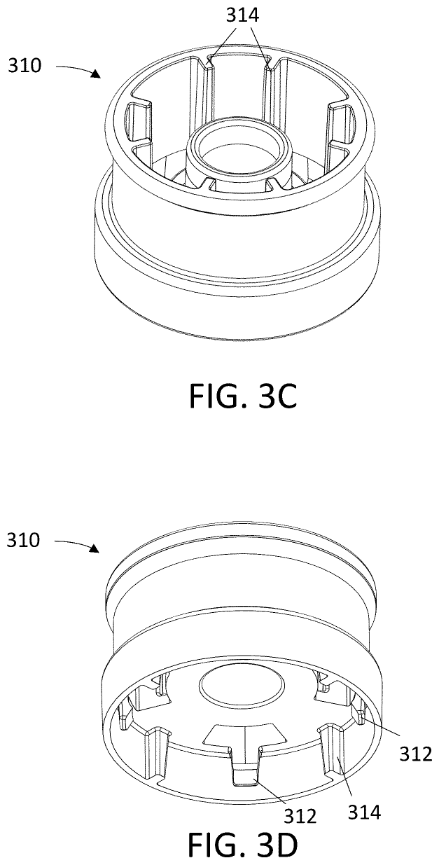

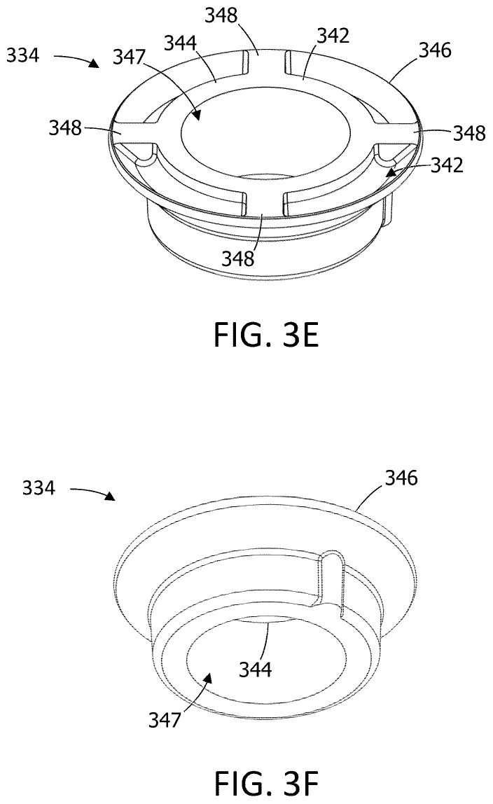

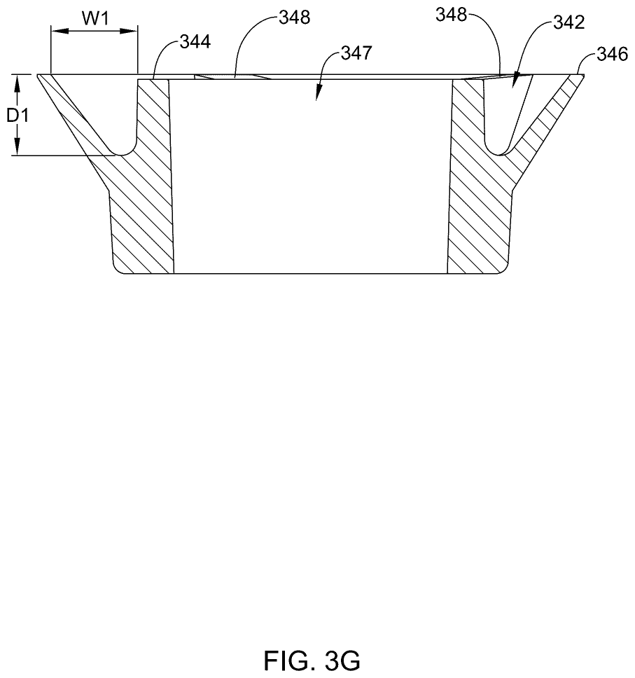

An example of a removable gas/water valve 300 is illustrated in FIGS. 3A-3E. as depicted in the perspective view and exploded view of FIG. 3A, the valve 300 may include a valve cap 302 with a gas hole 304, a valve collar 310, and a valve stem 320. A spring member 330 may extend from the valve cap 302 to the valve collar 310, while seals 332-336, depicted for example in FIG. 3B, may be disposed along an outside surface of the valve stem 320. To open the valve 300, a user may press downward on the valve cap 302, which moves the cap 302 and stem 320 relative to the collar 310. When the cap 302 is released, the spring member 330 returns the valve 300 to a biased or default position.

The valve stem 320 may have any suitable configuration configured to adjust positions within a valve well, adjust flow paths to the gas and water supplies and feeds, and couple to the cap 302. The valve stem 320 may couple to the cap 302 in any suitable manner. In some cases, a portion (e.g., a proximal portion) of the valve stem 320 extending proximally of the gas escape hole 304 may be coupled to the cap 302 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms.

The valve 300 may be formed in any suitable manner. In some cases, though not required, the valve 300 may be formed using a molding process, an injection molding process, a casting process, a finishing process, sanding, and/or by or with one or more additional or alternative manufacturing techniques. In one illustrative example, the valve 300 may be formed using an injection molding process.

FIG. 3B depicts a schematic exploded view of the valve stem 320 and the seals 332-336. As depicted in FIG. 3B, the valve stem 320 may include one or more gas inlet openings 322, one or more proximal openings 303, and one or more lumens 324 extending between the one or more gas inlet openings and the one or more proximal openings 303. The seals 332-336 may include an upper seal 332, a one-way seal 334, a lower seal 336, and/or one or more additional or alternative seals.

The valve stem 320 and the seals 322-326 and 408 may be formed from any suitable materials. In some cases, the valve stem 320 may be formed from a first material and the seals 322-326 and 408 may be formed from a second material, where the second material may be the same as or different than the first material. The valve stem 320 may be formed from a hard or rigid polymer and the seals 322-326 and 408 may be formed from a flexible polymer, but this is not required. In one example, the valve stem 320 may be formed from one or more of polymer, acrylonitrile butadiene styrene (ABS), polycarbonate, and/or other suitable materials. In another example, the seals 322-326 and 408 may be formed from one or more of a polymer, thermoplastic elastomers (TPE), thermoplastic polyurethane (TPU), liquid silicone rubber (LSR), and/or other suitable materials. In some examples, the valve stem 320 may be a single piece of injection-molded plastic. In some examples, the rigid thermoplastic used to form the valve stem 320 may drive certain design elements for the shape and size of valve components. For example, the central lumen 324 of the valve stem 320 may be slightly tapered to better accommodate injection molding. The structure and design of valve components may similarly be the result of the material choice and manufacturing process.

The material of the seals 322-326 and 408 may have any suitable durometer. In one example, the material of the seals 322-326 and 408 when formed on the valve stem 320 may have a durometer in a range of about 20-80 shore A, about 30-60 shore A, and/or other suitable values within one or more other suitable ranges of durometer, but could be softer or firmer depending on the geometry used for the seals and the amount of interference desired with the inner wall of the valve well 402. In one example, the seals 322-326 and 408 may be formed from silicone with a durometer in a range of 40-50 shore A, but this is not required.

The valve collar 310 may attach to a valve body and remain stationary relative to the valve body when the valve is inserted into the valve body and actuated. The cap 302, stem 320, and seals 332-336 may move downward within a valve well of the valve body when the valve 300 is actuated. Actuating the valve 300 may compress the spring 330 between the cap 302 and the collar 310. The spring 330 presses upward to return the valve 300 to its upward position when it is no longer pressed down.

As shown in FIG. 3D, the underside of the valve collar 310 may include a plurality of latches 312 spaced radially between a plurality of ribs 314. In some implementations, the latches 312 resiliently press against a lip of the endoscope to clip the collar 310, and consequently the valve 300, in place when installed.

FIGS. 3E and 3F depict schematic top and bottom perspective views of an illustrative configuration of a one-way seal 334 (e.g., a second seal). In some examples, the one-way seal 334 may be positioned around the valve stem 320 in a valve well so as to prevent fluid from a gas outlet from backflowing into a gas inlet.

As depicted in FIG. 3E, the one-way seal 334 may define a ravine 342. The ravine 342 may be defined by a stem engagement portion 344 and a seal portion 346, where the stem engagement portion 344 and the seal portion 346 are spaced (e.g., radially) from one another at a proximal end of the ravine 342 and converge and engage one another at or toward a distal end of the ravine 342. In some examples, one or more extensions 348 may extend between the stem engagement portion 344 and the seal portion 346 at one or more circumferential locations along the ravine 342. The stem engagement portion 344 may define a lumen 347 configured to receive the valve stem 320 and engage and/or couple with the valve stem 320 of the valve 300. The seal portion 346 may be configured to seal against an inner surface or wall of the valve well to prevent fluid from the gas outlet from backflowing into the gas inlet.

FIG. 3G depicts a schematic cross-section view of the illustrative one-way seal 334 depicted in FIGS. 3E and 3F. As depicted in FIG. 3G, the ravine 342 may be defined by and/or extend from the stem engagement portion 344 to the seal portion 346. The ravine 342 may have any suitable dimensions. In some examples, the ravine 342 of the illustrative one-way seal 334 depicted in FIG. 3G may have a maximum width W1 and a maximum depth of D1, but other suitable configurations are contemplated. In some examples, a proximal end of the seal portion 346 may extend to an axial position proximal of an axial position of a proximal end of the stem engagement portion 344 (e.g., as depicted in FIG. 3B) or the proximal end of the seal portion 346 may extend to an axial position equal to or distal of an axial position of the proximal end of the stem engagement portion 344.

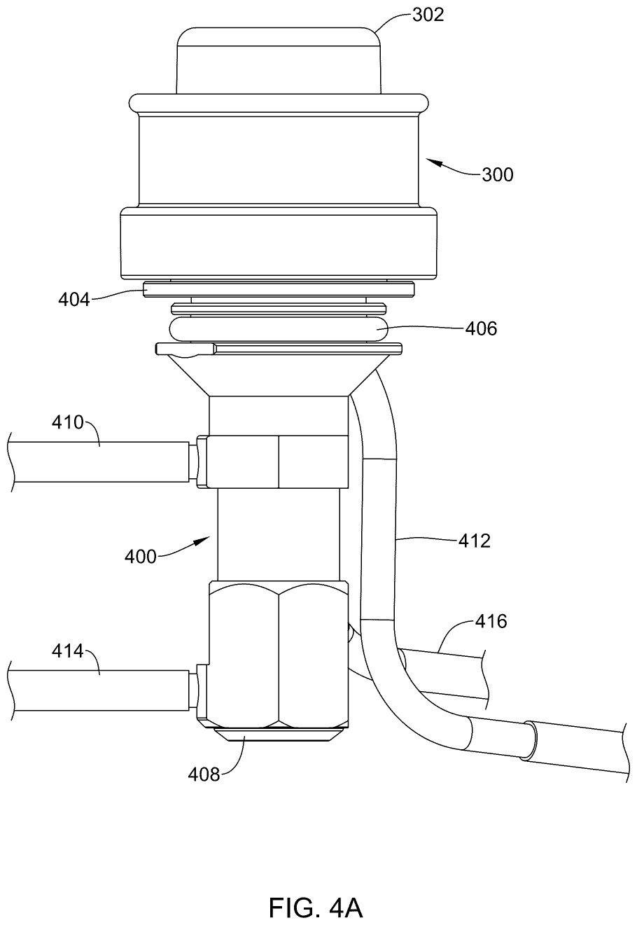

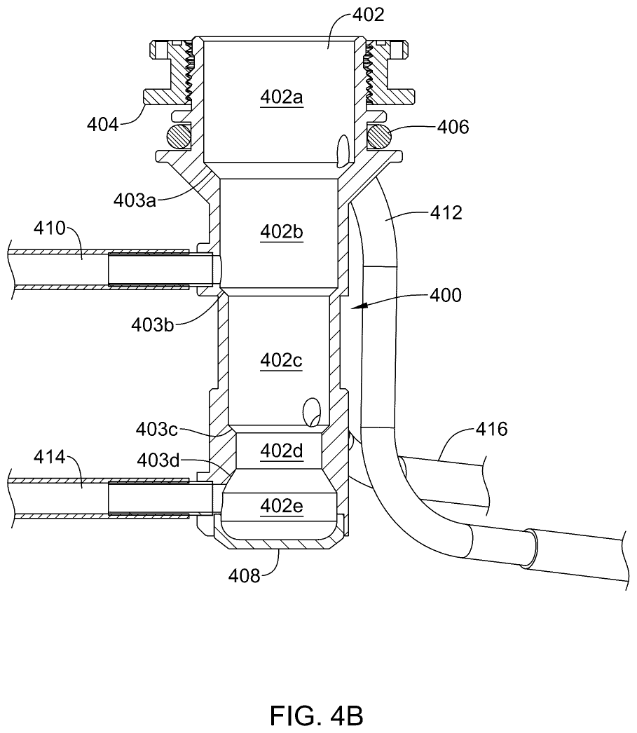

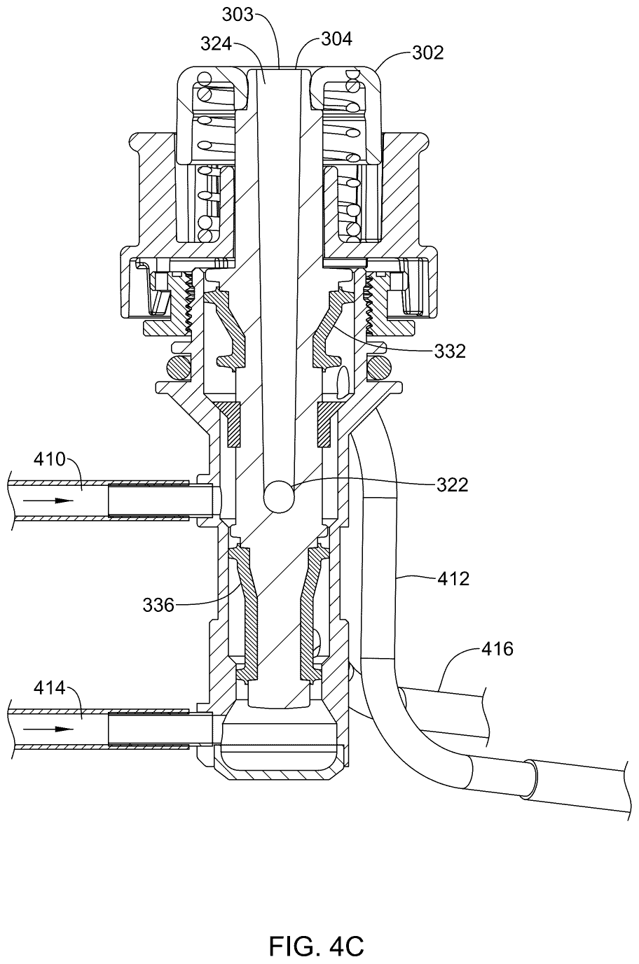

The valve 300 may be inserted into a valve body 400, as depicted for example in FIGS. 4A-4C, such as one of the locations 135 described above and illustrated in FIGS. 1 and 2. The valve body 400 may include an internal valve well 402 (e.g., see FIGS. 4B and 4C) sized and shaped to receive the stem 320 of the valve 300, as well as alternative valve designs (including each of those illustrated and described below). The valve body 400 may include a gas inlet passage 410 communicating with a source of gas as described above with respect to gas supply line 240a. A gas outlet passage 412 similarly communicates with a gas feed line 240b.

FIG. 4A shows the valve 300 attached to a valve body 400, which as noted above may be part of the endoscopic handle 115. A nut 404 and O-ring 406 may be along an outside surface of the valve body 400, while a body seal 408 may be at a bottom of the body 400 to seal the lower end of the internal valve well 402. As shown in FIG. 4B, the valve well 402 may be divided into internal chambers 402a-e separated by contours 403a-d, each of which corresponds to a change in the diameter of the internal side wall of the valve well 402. A first chamber 402a may have a first internal diameter extending from the top of the valve well 402 and accommodates the gas outlet passage 412. Moving downward, contour 403a reduces the side wall to a second smaller diameter of a second chamber 402b that includes the gas inlet passage 410. Contour 403b then further reduces the side wall to a third diameter associated a third chamber 402c, communicating with the water outlet passage 416. The contour 403c reduces the side wall to its fourth, smallest diameter at a fourth chamber 402d, after which the contour 403d increases the diameter at the fifth chamber 402e for the portion of the side wall where the water inlet 414 is located. The bottom portion of the well 402 has a further increased diameter to accommodate the body seal 408.

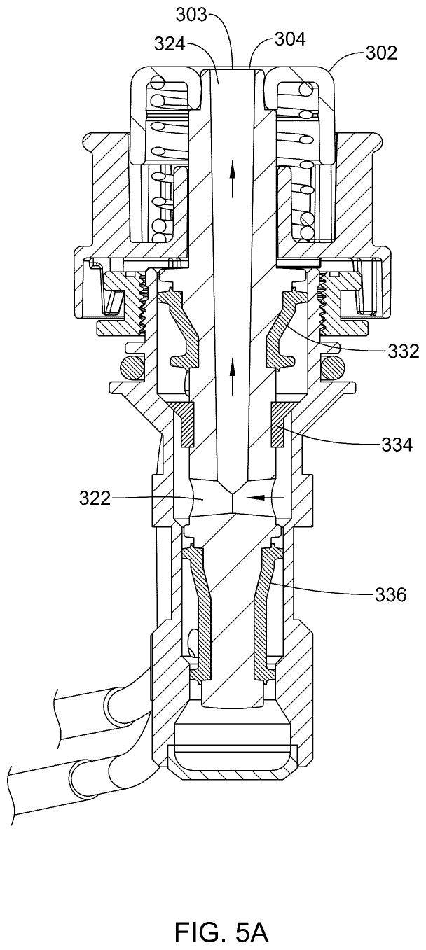

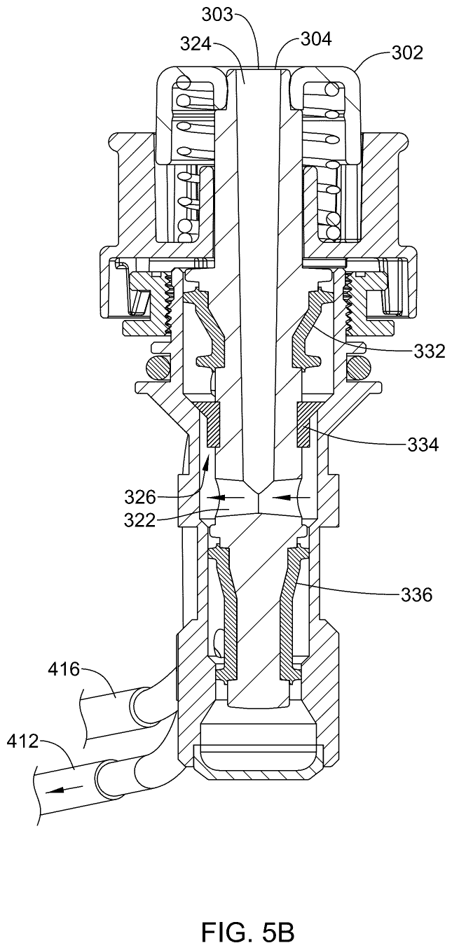

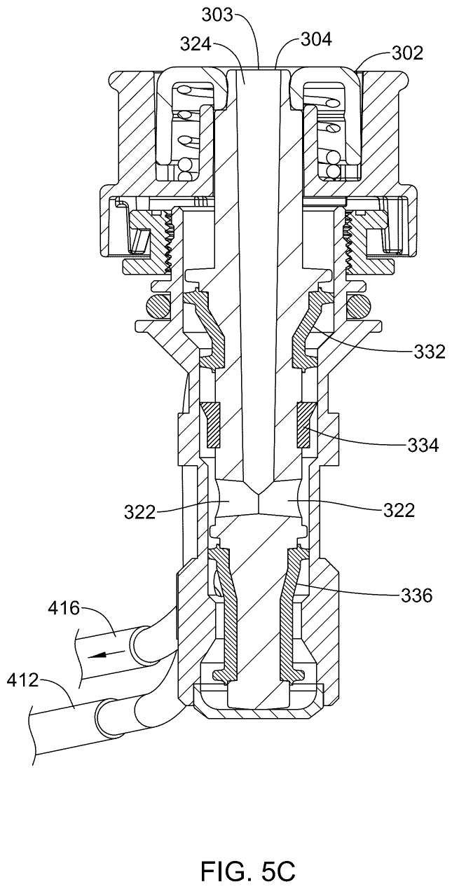

As shown in FIG. 4C as well as FIGS. 5A-5C, the valve stem 320 and the seals 332-334 surrounding the outer surface of the valve stem 320 and may be shaped to correspond to the internal diameters of the valve well 402 along its length. In some examples, the external diameter of the valve stem 320 may taper along its length such that a gap between the valve stem 320 outer surface and the inner surface of the valve well 402, as measured by the difference in diameter of the outer surface of the valve stem 320 and the inner surface of the valve well 402, is substantially the same at each point where a seal wiper is positioned to contact the well 402 inner surface. For this reason, the diameters of the four wipers associated with the two wiper seals 332 and 336 may be equal, but other suitable diameters of the four wipers are contemplated.

When the valve 300 is in the upward position illustrated in FIGS. 4C, 5A, 5B (e.g., in a first or second configuration), the top wiper of the upper wiper seal 332, and both wipers of the lower wiper seal 336 are in contact with the inner surface of the valve well 402. The diameters of the portions of the valve stem 320 at these three positions correspond, minus a set distance representing a constant spacing, to the diameters of the first, third, and fourth chambers 302a, 302c, and 302d where each of the wipers is positioned. Similarly, when the valve 300 is in the lower position illustrated by FIG. 5C (e.g., in a third configuration), the two wipers of the upper wiper seal 332 and the upper wiper of the lower wiper seal 336 correspond, minus the same set distance, to the diameters of the first, second, and fourth chambers 302a, 302b, and 302d where the wipers are positioned.

FIG. 5A shows an open or first configuration for the valve when the proximal opening 303 of the valve stem 320 and the gas escape hole 304 in the cap 302 is unblocked. The valve stem 320 may include the proximal opening 303 and two gas inlets 322 connected to a central lumen 324, which opens into the gas escape hole 304. Gas passes through the gas inlets 322, up the central lumen 324, and out into the room. This is may be a biased or default configuration when a user is not actively manipulating the valve 300. In this position, the upper wiper of the upper wiper seal 332 blocks gas from exiting the chamber 402a through the top of the valve well 402, while the one-way valve 334 does not block gas from moving from the second chamber 404b to the first chamber 404a. Furthermore, water entering the water inlet passage 414 is contained within the fourth and fifth chambers 402d and 402e by the lower wiper of the lower wiper seal 336.

FIG. 5B shows a second configuration for the valve 300 in which the gas escape hole 304 is obstructed. In some implementations, the user may place a finger over the hole 304. A flap or other device may also be included for placement over the gas escape hole 304 in other embodiments. When the gas escape hole 304 is obstructed, gas instead flows through a path defined by an exterior recess 326 between an exterior surface of the valve stem 320 and the internal side wall of the valve well 402. Gas passes through the gas inlet passage 410, through the exterior recess 326, through the gas outlet passage 412, and into the endoscope for use in insufflation as described.

Three seals 332, 334, and 336 may surround the valve stem 320 along its length. An upper wiper seal 332 may be disposed below the valve cap 302 and above the exterior recess 326, obstructing flow in the valve well above the location of the gas outlet passage 412. A one-way seal 334 distal of the upper wiper seal 332 may intersect the exterior recess 326 between the valve stem 320 and the valve well, permitting gas to flow upwards towards the gas outlet passage 412 but not downwards towards the gas inlet passage 410. A lower wiper seal 336 located distal of the one-way seal 334 may include two wiper flanges. Each of the seals 332, 334, 336 may be a flexible material with lower hardness than that of the valve components, such as silicone.

The valve body 400 includes the water inlet passage 414 connected to a water supply, and the water outlet passage 416 connected to a water feed line. When the valve 300 is in the upper position as in FIGS. 5A and 5B, the lower of the two wipers of the lower seal 336 may sit above the water inlet passage 414, obstructing water from proceeding up the valve well or into the water outlet passage 416.

FIG. 5C shows a third configuration in which the valve 300 is positioned lower in the valve body 400. Downward force on the valve cap 302 may cause and maintain this position. When the valve cap 302 is released, the spring member 330 returns the valve 300 to its previous position. In this configuration, the exterior recess 326 is no longer aligned with the gas inlet passage 410 and gas outlet passage 412 in the valve body 400. The one-way seal 324 may be seated along the inner wall of the valve body 400 to obstruct gas flow above the gas inlet passage 410. The two wiper flanges of the lower wiper seal 326 are, in this configuration, positioned above the water outlet passage 416 and below the water inlet passage 414, creating an annular passage between the valve stem 320 and valve body 400 in which water can flow from the water inlet passage 414 to the water outlet passage 416. Upon release of the downward force and return of the valve cap 302 to its previous position, the placement of the lower seal 336 again prevents additional water from entering the feed through the annular passage.

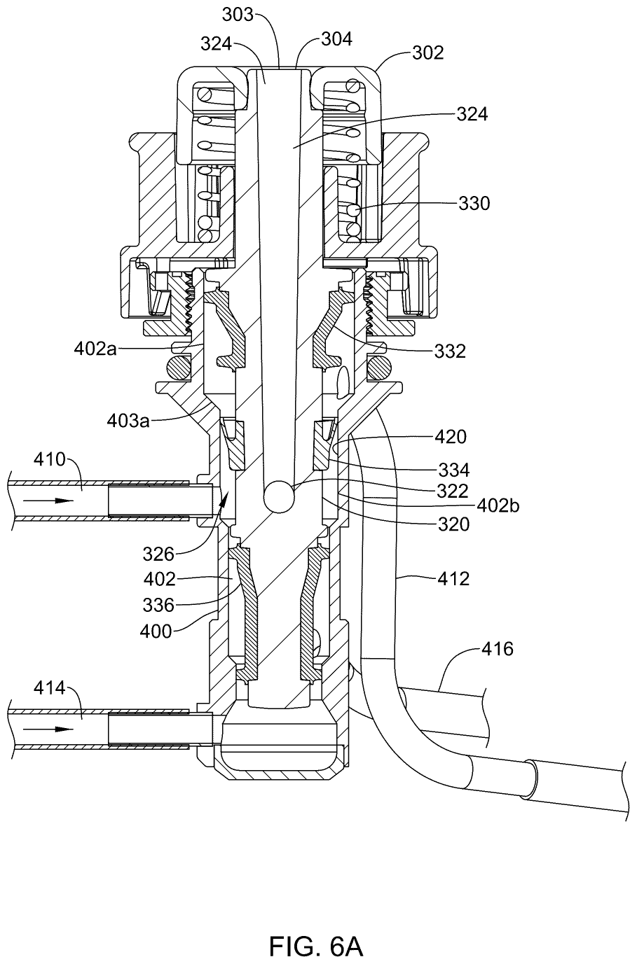

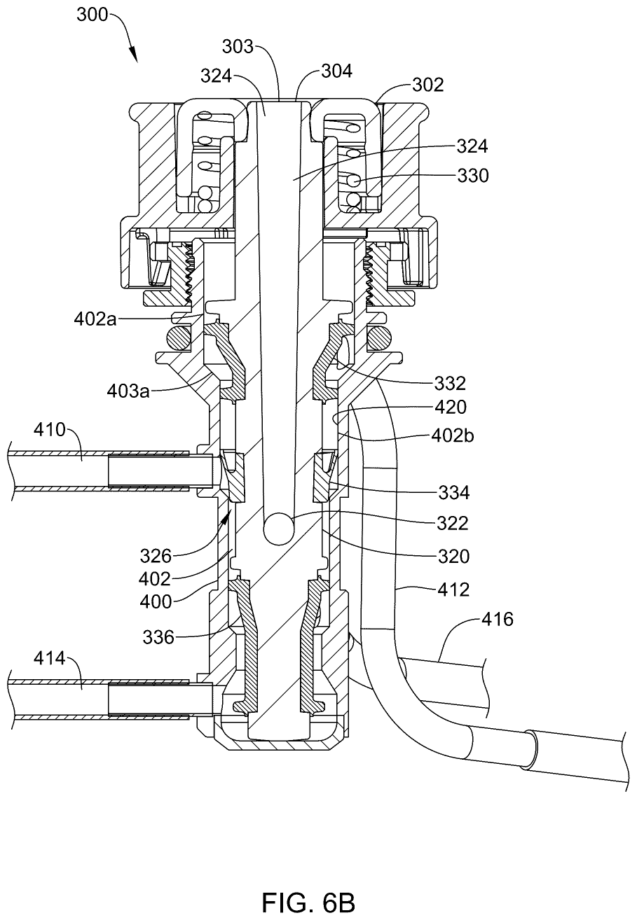

FIGS. 6A and 6B depict an illustrative configuration of the valve stem 320 and the seals 332-336 surrounding an outer surface of the valve stem 320. The seal 332 and the seal 336 may be configured similar to as discussed with respect to FIGS. 4A-5C. The one-way seal 332 may have different configuration than depicted in FIGS. 4A-5C relative to the valve stem 320 and/or the inner surface of the valve well.

As depicted in FIG. 6A, the one-way seal 334 may be located along the valve stem 320 at a position distal of where the one-way seal 334 is positioned along the valve stem in the configurations of FIGS. 4A-5C. For example, when the valve 300 is in the first configuration or the second configuration with valve 300 in a resting (e.g., biased) state and the gas escape hole 304 (e.g., a proximal opening) is or is not covered, the one-way seal 334 may be configured to seal against a vertical wall 420 (e.g., a wall parallel with a longitudinal axis of the lumen 324) of inner surface of the valve well 402 (e.g., at the second chamber 402b) between the gas inlet passage 410 and the gas outlet passage 412 to prevent fluid from traveling to the gas inlet from the first chamber 402a. In some examples, the location at which one-way seal 334 may seal against the inner surface of the valve well 402 may be distal of a transition (e.g., distal of the contour 403a) between an inner diameter of the first chamber 402a an inner diameter of the second chamber 402b, as depicted for example in FIG. 6A.

FIG. 6B depicts the valve 300 in the third configuration in which the valve 300 is positioned lower in the valve well 402. Downward force on the valve cap 302 causes and maintains valve 300 in the lower position valve well 402. When the valve cap 302 is released, the spring member 330 returns the valve 300 to the first configuration or the second configuration (e.g., as depicted in FIG. 6A). In the second configuration, the one-way seal 334 may be seated along the vertical wall 420 of the valve well 402 to prevent or obstruct fluid flow from above the gas inlet passage 410 from entering the gas inlet passage 410.

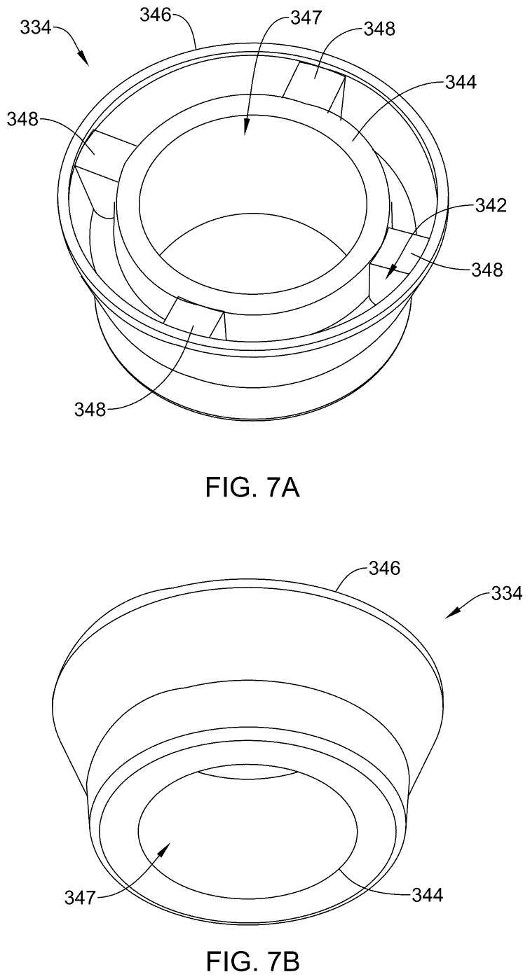

FIGS. 7A and 7B depict schematic top and bottom perspective views of an illustrative configuration of a one-way seal 334. In some examples, the one-way seal 334 may be configured as a one-way valve and positioned around the valve stem 320 in a valve well so as to prevent fluid from a gas outlet from backflowing into a gas inlet.

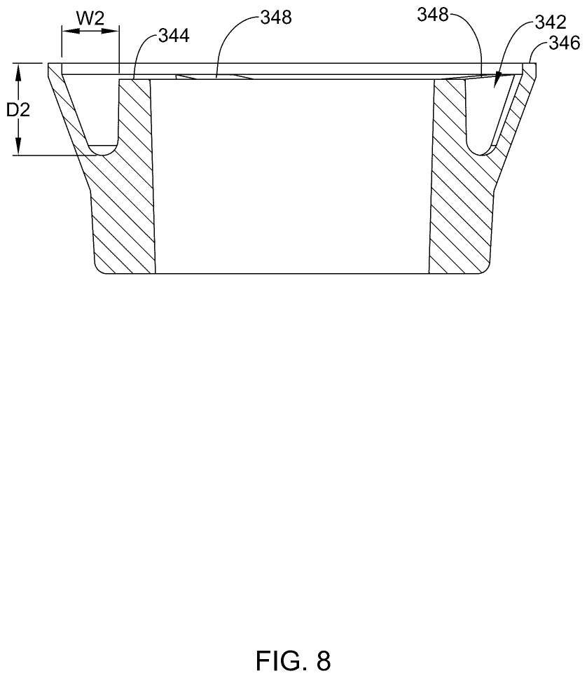

Similar to the configuration of the one-way seal 334 depicted in FIGS. 3E and 3F, the one-way seal 334 depicted in FIG. 8 may define the ravine 342. The ravine 342 may be defined by the stem engagement portion 344 and the seal portion 346, where the stem engagement portion 344 and the seal portion 346 are spaced (e.g., radially spaced) from one another at the proximal end of the ravine 342 and converge and engage one another at or toward the distal end of the ravine 342. In some examples, one or more extensions 348 may extend between the stem engagement portion 344 and the seal portion 346 at one or more circumferential locations along the ravine 342. The stem engagement portion 344 may define the lumen 347 configured to receive the valve stem 320 and engage and/or couple with the valve stem 320. The seal portion 346 may be configured to seal against an inner surface or wall of the valve well to prevent fluid from the gas outlet from backflowing into the gas inlet.

As depicted in FIG. 7A, the ravine 342 of the one-way seal 334 may be deeper and narrower than the ravine 342 of the one-way seal 334 depicted in FIGS. 3E and 3F. The ravine 342, however, may have other suitable configurations.

FIG. 8 depicts a schematic cross-section view of the illustrative one-way seal 334 depicted in FIGS. 7A and 7B. As depicted in FIG. 8, the ravine 342 may be defined by and/or extend between the stem engagement portion 344 and the seal portion 346. In some examples, a proximal end of the seal portion 346 may extend to an axial position proximal of an axial position of a proximal end of the stem engagement portion 344 (e.g., as depicted in FIG. 8) or the proximal end of the seal portion 346 may extend to an axial position equal to or distal of an axial position of the proximal end of the stem engagement portion 344.

The ravine 342 may have any suitable dimensions. In some examples, the ravine 342 of the illustrative one-way seal 334 depicted in FIG. 8 may have a maximum width W2 and a maximum depth of D2, where the maximum width W2 of the ravine 342 depicted in FIG. 8 may be less than the width W1 of the ravine 342 depicted in FIG. 3G and the depth D2 of the ravine 342 depicted in FIG. 8 may be greater than the depth D1 of the ravine 342 depicted in FIG. 3G. In other terms, the configuration of the ravine 342 depicted in FIG. 8 may be deeper and skinnier (e.g., less wide) than the ravine 342 depicted in FIG. 3G. Other suitable configurations of the ravine 342 are contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve assembly for a medical device, comprising:
a valve body having an internal valve well, the valve body having a gas inlet passage, a gas outlet passage, a water inlet passage, and a water outlet passage each connected to the internal valve well, the internal valve well comprising:
a first chamber having a first diameter, the first chamber opening into the gas outlet passage,
a second chamber below the first chamber and having a second diameter smaller than the first diameter, the second chamber opening into the gas inlet passage,
a third chamber below the second chamber and having a third diameter smaller than the second diameter, the third chamber opening into the water outlet passage,
a fourth chamber below the third chamber and having a fourth diameter smaller than the third diameter, and
a fifth chamber below the fourth chamber and having a fifth diameter larger than the fourth diameter, the fifth chamber opening into the water inlet passage;
a valve cap having a gas hole;
a valve stem connected to the valve cap and configured to translate within the valve well between an upper position and a lower position, the valve stem comprising:
two gas inlets, and
a central lumen extending from the gas inlets in the valve stem to the gas hole in the valve cap;
an upper wiper seal extending circumferentially around the valve stem, the upper wiper seal comprising first and second wipers positioned such that,
when the valve seal is in the upper position, the first wiper contacts the internal wall of the first chamber of the valve well to obstruct gas flow through the first chamber, and,
when the valve seal is in the lower position, the second wiper contacts the internal wall of the second chamber of the valve well to obstruct gas flow through the second chamber;
an lower wiper seal extending circumferentially around the valve stem, the lower wiper seal comprising first and second wipers positioned such that,
when the valve seal is in the upper position, the second wiper contacts the internal wall of the fourth chamber of the valve well to obstruct water flow through the fourth chamber, and
when the valve seal is in the lower position, the first wiper contacts the internal wall of the fourth chamber of the valve well to obstruct water flow through the fourth chamber.

2. The valve assembly of claim 1, further comprising a one-way seal extending circumferentially around the valve stem, the one-way seal contacting the internal wall of the valve well to obstruct gas flow downwards into the second chamber while allowing gas to flow upwards.

3. The valve assembly of claim 2, wherein the one-way seal is positioned in contact with a contour of the valve well between the first and second chambers of the valve well when the valve stem is in the upper position, the one-way seal obstructing gas flow from the first chamber into the second chamber.

4. The valve assembly of claim 2, wherein the one-way seal is a flexible seal formed of a material with a durometer less than the durometer of a material forming the valve stem.

5. The valve assembly of claim 2, wherein the one-way seal is configured to contact and seal against a vertical portion of the internal wall of the valve well at the second chamber.

6. The valve assembly of claim 1, wherein the upper wiper seal is a flexible seal formed of a material with a durometer less than the durometer of a material forming the valve stem.

7. The valve assembly of claim 1, wherein the lower wiper seal is a flexible seal formed of a material with a durometer less than the durometer of a material forming the valve stem.

8. The valve assembly of claim 1, wherein the valve stem is injection-molded thermoplastic.

9. The valve assembly of claim 1, wherein the upper and lower wipers of the upper wiper seal and the upper and lower wipers of the lower wiper seal are all of equal width.

10. The valve assembly of claim 1, wherein the valve stem includes portions having varying diameters, wherein the diameters of the portions vary according to the varying diameters of the first, second, third, and fourth chambers of the valve well.

11. The valve assembly of claim 1, further comprising:
a valve collar configured to attach to the valve body, the valve collar remaining stationary relative to the valve body when the valve stem moves between the upper and lower positions; and
a spring member connected to the valve collar and the valve cap, the spring member biased to move the valve cap upward to return the valve stem to the upper position from the lower position.

12. The valve assembly of claim 11, wherein the valve collar comprises a plurality of latches sized and positioned to press against the valve body when the valve collar is attached to the valve body.

13. The valve assembly of claim 11, wherein the valve collar is injected- molded thermoplastic.

14. The valve assembly of claim 11, wherein the spring member is formed of a material with a durometer less than the durometer of a material forming the valve collar.

15. An endoscopic medical device, comprising:
an endoscopic probe; and
the valve assembly coupled to the endoscope for use in an endoscopic procedure, the valve assembly comprising:
a valve body having an internal valve well, the valve body having a gas inlet passage, a gas outlet passage, a water inlet passage, and a water outlet passage each connected to the internal valve well, the internal valve well comprising:
a first chamber having a first diameter, the first chamber opening into the gas outlet passage,
a second chamber below the first chamber and having a second diameter smaller than the first diameter, the second chamber opening into the gas inlet passage,
a third chamber below the second chamber and having a third diameter smaller than the second diameter, the third chamber opening into the water outlet passage,
a fourth chamber below the third chamber and having a fourth diameter smaller than the third diameter, and
a fifth chamber below the fourth chamber and having a fifth diameter larger than the fourth diameter, the fifth chamber opening into the water inlet passage;
a valve cap having a gas hole;
a valve stem connected to the valve cap and configured to translate within the valve well between an upper position and a lower position, the valve stem comprising:
two gas inlets, and
a central lumen extending from the gas inlets in the valve stem to the gas hole in the valve cap;
an upper wiper seal extending circumferentially around the valve stem, the upper wiper seal comprising first and second wipers positioned such that,
when the valve seal is in the upper position, the first wiper contacts the internal wall of the first chamber of the valve well to obstruct gas flow through the first chamber, and,
when the valve seal is in the lower position, the second wiper contacts the internal wall of the second chamber of the valve well to obstruct gas flow through the second chamber;
a lower wiper seal extending circumferentially around the valve stem, the lower wiper seal comprising first and second wipers positioned such that,
when the valve seal is in the upper position, the second wiper contacts the internal wall of the fourth chamber of the valve well to obstruct water flow through the fourth chamber, and
when the valve seal is in the lower position, the first wiper contacts the internal wall of the fourth chamber of the valve well to obstruct water flow through the fourth chamber.

16. The endoscopic medical device of claim 15, the valve assembly further comprising a one-way seal extending circumferentially around the valve stem, the one- way seal contacting the internal wall of the valve well to obstruct gas flow downwards into the second chamber while allowing gas to flow upwards.

17. The endoscopic medical device of claim 16, wherein the one-way seal is positioned in contact with a contour of the valve well between the first and second chambers of the valve well when the valve stem is in the upper position, the one-way seal obstructing gas flow from the first chamber into the second chamber.

18. The endoscopic medical device of claim 16, wherein the one-way seal is positioned in contact with a vertical inner wall of the valve well between the gas inlet passage of the valve body and the gas outlet passage of the valve body.

19. A valve for a medical device, the valve comprising:
a valve stem configured to translate within a valve well between a first configuration and a second configuration, the valve stem comprising:
a proximal opening;
a gas inlet; and
a central lumen extending from the gas inlet to the gas escape hole; and
a one-way seal extending circumferentially around the valve stem at an axial location along the valve stem distal of the proximal opening and proximal of the gas inlet, and
wherein the one-way seal is configured to contact a vertical inner wall of a valve well between a gas inlet of the valve well and a gas outlet of the valve well.

20. The valve of claim 19, further comprising:
an upper wiper seal extending circumferentially around the valve stem at an axial location proximal of the one-way seal, the upper wiper seal comprising first and second wipers; and
a lower wiper seal extending circumferentially around the valve stem at an axial location distal of the one-way seal, the lower wiper seal comprising first and second wipers.
